(19) United States
(12) Patent Application Publication
(10) Pub. No.: US 2023/0207100 A1
(43) Pub. Date: Jun. 29, 2023

(54) NUTRITIONAL INFORMATION EXCHANGE SYSTEM

(71) Applicant: Baystride, Inc., Leawood, KS (US)

(72) Inventors: John PHELPS, Point Roberts, WA (US); Dan PITTS, Leawood, KS (US); Darryl RAWLINGS, Seattle, WA (US)

(21) Appl. No.: 17/940,756

(22) Filed: Sep. 8, 2022

Related U.S. Application Data
(63) Continuation of application No. PCT/US2021/021976, filed on Mar. 11, 2021.
(60) Provisional application No. 62/988,864, filed on Mar. 12, 2020.

(51) Int. Cl. G16H 20/60; G06Q 50/04; G06Q 10/087; G16H 40/67; G16H 50/20

(52) U.S. Cl. CPC G16H 20/60; G06Q 10/087; G06Q 50/04; G16H 40/67; G16H 50/20

(57) ABSTRACT

The present disclosure provides methods and systems for nutritional information exchange. The system may comprise: a backend component implemented on a computer, and the backend component is configured to: (a) receive data from a system in a veterinary practice, and the data comprises information about one or more characteristics of an animal and a nutrition goal; (b) create a nutritional plan based on the data, and the nutritional plan includes a set of meals each comprising one or more elements varied based at least in part on a type of meal; (c) select a combination of ingredients for each of the set of meals based on (i) available ingredients received from a manufacture system and the (ii) one or more elements for each of the set of meals; and (d) transmit the combination of ingredients for each of the set of meals to the manufacture system for fabrication and distribution.

### CROSS-REFERENCE

This application is a continuation of International Patent Application No. PCT/US2021/021976, filed Mar. 11, 2021, which claims priority to U.S. Provisional Pat. Application No. 62/988,864, filed Mar. 12, 2020, each of which is entirely incorporated herein by reference.

### FIELD

The disclosure relates generally to information exchange system for pet nutritional planning and more particularly to systems and methods for producing individually packaged pet meals that are tailored to an individual animal.

### BACKGROUND

Animal nutrition is based on one-size-fits-most packaging wherein food is manufactured on a large scale and animal caretakers feed their animals this food for every meal of the day. Some manufactures make foods for certain types of dogs or dogs in certain age ranges or with certain conditions. Generalized animal nutrition is less than ideal and can lead to animals receiving inappropriate, excessive and/or unbalanced nutrition.

### SUMMARY

Systems, apparatus, and methods for personalized animal nutrition are disclosed. In particular, the present disclosure provides an information exchange system with improved traffic control and data integration to enable efficient communication channels among veterinary practice entities, pet owners, meal manufacturing systems, and the like. The nutritional planning and individualized meals provided herein allow veterinarians to provide effective oversight to the nutrition process and for individualized control of an animal’s nutritional intake on a meal-by-meal basis.

The present disclosure provides system for nutritional information exchange. The system comprises: a backend component implemented on a computer, wherein the backend component is configured to: (a) receive data from a system in a veterinary practice via a data integration agent, wherein the data comprises information about one or more characteristics of an animal and a nutrition goal; (b) create a nutritional plan based on the data, wherein the nutritional plan includes a set of meals each comprising one or more elements varied based at least in part on a type of meal; (c) select a portioned combination of ingredients for each of the set of meals based on (i) available ingredients received from a manufacture system and the (ii) one or more elements for each of the set of meals; and (d) transmit the portioned combination of ingredients for each of the set of meals to the manufacture system for fabrication and packaging the portioned combination of ingredients for each meal individually.

In some embodiments, the system in the veterinary practice comprises a graphical user interface for receiving the data. In some embodiments, the data integration agent is connected to the system in the veterinary practice, and wherein the data integration agent provides a data abstraction layer for enabling the backend component to access or retrieve the data from the veterinary practice.

In some embodiments, the one or more characteristics are extracted from the data using machine learning techniques. In some embodiments, the backend component is configured to further receive activity data from a motion tracking device to obtain a characteristic about an activity level of the animal. In some embodiments, the one or more characteristics of the animal comprise one or more of mature body weight, body condition score, muscle condition score, and ideal weight.

In some embodiments, the one or more elements comprise a nutritional value for a meal or series of meals. Alternatively, the one or more elements comprise a nutritional value for a partial meal.

Another aspect of the present disclosure provides a non-transitory computer readable medium comprising machine executable code that, upon execution by one or more computer processors, implements any of the methods above or elsewhere herein. In some embodiments, the one or more elements comprise a target nutritional value range for a period of time.

In some embodiments, the type of meal includes a morning meal, a midday meal, an evening meal, and a snack provided within a day, or a subset thereof. In some cases, the meal set includes packaged meals of different types to be provided within a day.

In some embodiments, the backend component is configured to further modify the nutritional plan based on a user input received from the system in the veterinary practice. In some embodiments, the backend component is configured to further modify the nutritional plan based on data received from a user device associated with an owner of the animal. In some cases, the data comprises an image of the animal.

In some embodiments, the nutritional plan is created, modified or supplemented using a machine learning algorithm trained model. In some embodiments, the combination of ingredients is selected using a machine learning algorithm trained model.

In a related yet separate aspect, a method for nutritional information exchange is provided. The method comprises: (a) receiving data from a system in a veterinary practice via a data integration agent, wherein the data comprises information about one or more characteristics of an animal and a nutrition goal; (b) creating a nutritional plan based on the data, wherein the nutritional plan includes a set of meals each comprising one or more elements varied based at least in part on a type of meal; (c) selecting a combination of ingredients for each of the set of meals based on (i) available ingredients received from a manufacture system and (ii) the one or more elements for each of the set of meals; and (d) transmiting the portioned combination of ingredients for each of the set of meals to the manufacture system for fabrication and packaging the portioned combination of ingredients for each meal individually.

In some embodiments, the system in the veterinary practice comprises a graphical user interface for receiving the data. In some embodiments, the data integration agent is connected to the system in the veterinary practice, and wherein the data integration agent provides a data abstraction layer for enabling accessing or retrieving the data from the veterinary practice.

In some embodiments, the one or more characteristics are extracted from the data using machine learning techniques. In some embodiments, the method further comprises receiving activity data from a motion tracking device to obtain a characteristic about an activity level of the animal. In some embodiments, the one or more characteristics of the animal comprise one or more of mature body weight, body condition score, muscle condition score, and ideal weight.

In some embodiments, the one or more elements comprise a nutritional value for a meal. Alternatively, the one or more elements comprise a nutritional value for a partial meal. In some embodiments, the one or more elements comprise a target nutritional value range for a day, week, month or other period.

In some embodiments, the type of meal includes a morning meal, a midday meal, an evening meal, and a snack provided within a day, or a subset thereof. In some cases, the meal set includes packaged meals of different types to be provided within a day.

In some embodiments, the method further comprises modifying the nutritional plan based on a user input received from the system in the veterinary practice. In some embodiments, the method further comprises modifying the nutritional plan based on data received from a user device associated with an owner of the animal. In some cases, the data comprises an image of the animal.

In some embodiments, the nutritional plan is created using a machine learning algorithm trained model. In some embodiments, the combination of ingredients is selected using a machine learning algorithm trained model.

In another aspect, a system for nutritional information exchange is provided. The system comprises: nutritional management server in communication with one or more veterinary management systems and one or more manufacturing servers, wherein the nutritional management server comprises (i) a memory for storing a set of software instructions, and (ii) one or more processors configured to execute the set of software instructions to: (a) receive and exchange, via a data integration agent, nutritional data from the one or more veterinary management systems, wherein the nutritional data comprises information about one or more characteristics of a selected animal and a nutrition goal and at least a portion of the nutritional data is retrieved from a database operably coupled to the one or more veterinary management systems; b) create a nutritional plan based at least in part on the nutritional data, wherein the nutritional plan includes a set of meals each comprising one or more elements varied based at least in part on a type of meal; (c) select a portioned combination of ingredients for each of the set of meals based on (i) available ingredients received from the one or more manufacturing servers and the (ii) one or more elements for each of the set of meals; and (d) transmit the portioned combination of ingredients for each of the set of meals to the one or more manufacturing servers for production and packaging of the portioned combination of ingredients for each meal individually for the selected animal. In some embodiments, the nutritional management server further modifies the nutritional plan based on data received from at least one of a plurality of end node devices associated with the selected animal and an owner of the selected animal.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a method for individually planning an animal’s meals, in accordance with one or more embodiments herein.

FIG. 2 illustrates a system for individually planning an animal’s meals, in accordance with one or more embodiments herein.

FIG. 3 illustrates the backend system of FIG. 2 and/or FIG. 1, in accordance with one or more embodiments herein.

FIG. 4 illustrates the manufacturing and packaging system of FIG. 1 and/or FIG. 2, in accordance with one or more embodiments herein.

FIG. 5 illustrates a method of determining the nutrition for and packing individualized meals, in accordance with one or more embodiments herein.

FIG. 6 shows a network environment in which a nutritional information exchange system is implemented.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

In some cases, a patient, subject, or animal may also be referred to as pet. A patient refers to an animal being treated by a veterinary practice. As utilized herein, the term “veterinary practice” may refer to a hospital, clinic or similar where services are provided for an animal. A pet owner is a guardian of the pet and could be the pet owner, pet sitter, or similar pet guardian. A “medical professional,” as used herein, may include a medical doctor, a veterinarian, a medical technician, a veterinary technician, a medical researcher, a veterinary researcher, a naturopath, a homeopath, a therapist, or the like.

The disclosure may be applicable to a tracking device, system and method that is architected as described below and it is in this context that the disclosure will be described. It will be appreciated, however, that the device, system and method has greater utility, such nutrition beyond the veterinary setting, such as pet owner based nutrition, and for pets beyond the cats and dogs discussed herein. The systems and methods may be architected in other manners and using other mechanisms that are within the scope of the disclosure.

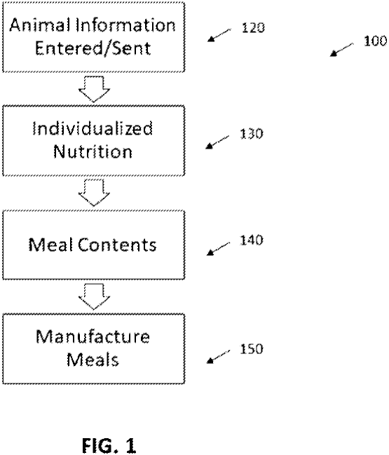

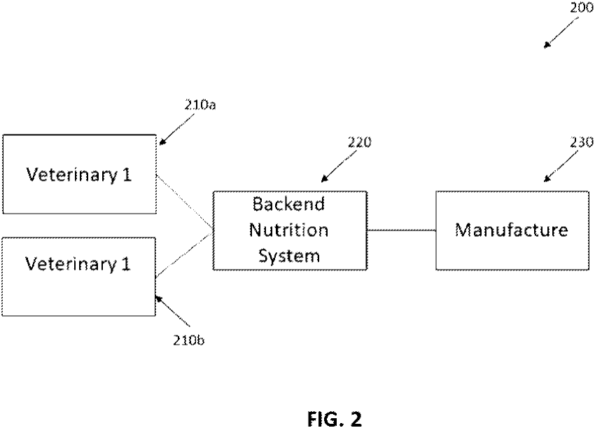

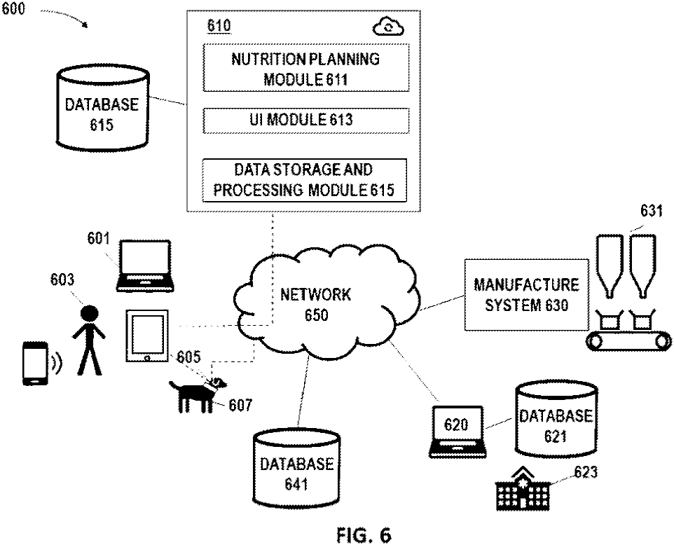

FIG. 1 illustrates a method 100 of determining the nutrition for and packing individualized meals. The method 100 may include receiving animal characteristics, determining a daily nutritional intake for the animal based on the animal characteristics, selecting individualized meal ingredients for each meal for the animal, and packaging the individualized meal ingredients into individual packages for each meal. The method may be carried out by a system, such as system 200, depicted in FIG. 2 or FIG. 6.

The method 100 may begin at block 120, where animal characteristics for an animal are received. At block 120, an animal’s characteristics may be received from a veterinary practice or from a pet owner. These animal characteristics may include the animal’s age, sex, breed, gender, species, weight, height, mature body weight, body condition score, muscle condition score, diseases, conditions, blood analysis results, medical history, current treatments, supplements and/or medications the animal is taking or prescribed, ideal weight, activity level and the like.

In some cases, the animal characteristics may be received in response to a request. For example, a request including animal name or a unique identifier may be used to retrieve the animal characteristics from a storage system or database.

In some embodiments, mobile or other telemedicine applications that use audio, stored video or live feeds maybe used to gather information about the pet’s characteristics such as the animal’s weight/condition and other characteristics described herein. The animal’s characteristics may be received from a pet owner, a pet owner’s recording system (such as a phone application that stores pictures or video). For example, a pet owner may input the animal characteristics via an application running on a pet owner’s device. Alternatively or additionally, at least a portion of the animal’s characteristics may be received from a health or motion tracking device carried by the animal. For example, data indicative of an activity level may be received from a motion tracking device carried by an animal.

In some cases, one or more animal’s characteristics may be received from a veterinarian, veterinarian staff member or veterinarian system, such as a veterinarian hospital’s practice management system 210a, depicted in FIG. 2. The veterinarian system 210a may include a practice information management system (PIMS), that includes the animal characteristics. The animal’s characteristics may be transmitted to a nutritional system for processing, such as the backend nutrition system 220, depicted in FIG. 2, to generate a nutritional plan. In some cases, at least portion of the animal characteristics may be provided by a medical professional through a veterinarian system. For example, a veterinarian or veterinarian professional, such as a veterinarian technician or other professional involved in the care and treatment of an animal collects or enters the information in their veterinary practice information management system, which may be part of the veterinary system 210. The veterinary practice information management system (PIMS) may provide the animal characteristics to the backend and nutritional system 220, depicted in FIG. 2. In some embodiments, each veterinary system may comprise a respective data integration agent, a user interface (e.g., browser, camera, etc.) for receiving input (e.g., modification of pet food, nutrition goal, information or medical record about an animal, picture or video of an animal, etc.) from a medical professional, and a communication module to enable communication and data transmission with a backend nutritional system for integration and further analysis. Details about the data integration agent and nutritional system are described later herein.

In alternative embodiments, one or more of the animal characteristics may be obtained from collection of biological samples. For instance, biological samples of an animal may be collected and analyzed to obtain a health status of the animal. The biological samples may include, but is not limited to, at least one of stool, hair, blood, microbiome, saliva, tissue, urine, advanced glycation end products (AGEs) levels, and DNA. The biological samples may be collected at a veterinary practice, or shipped in a kit. The biological sample analysis may be performed at the veterinary practice or by an entity that is in communication with the back end nutritional system. The biological sample may be collected prior to generating a nutritional plan. Alternatively, the biological sample may be collected and analyzed to adjust a nutritional plan. For example, biological analysis may be performed to determine a pet’s individual reaction to a diet and the pet’s ability to change its health status/condition (including, but not limited to weight, activity level, stool quality, immune status, oral/dental health, skeletal health, skin and coat health, etc.), which may be different than a reaction of another pet in the same category to the same diet.

At block 130, an individualized nutritional plan for each animal is determined. The individualized nutritional plan may be determined based on the characteristics of the animal. The characteristics of the animal may include historic, current and anticipated future characteristics derived from the empirical data. In some cases, the individualized nutritional plan may also be determined based on known nutritional guidelines for particular species and breeds of animals or based on the experience and recommendation of a veterinary hospital.

The individualized nutritional plan may include one or more elements. For example, the individualized nutritional plan may include total caloric intake, total number of calories from one or more carbohydrates, total number of calories from one or more fats, total number of calories from one or more proteins, total number of calories from one or more vegetables, total number of calories from one or more fruits, total number of grams of one or more fats, total number grams of one or more proteins, total number of grams of one or more vegetables, total number of grams of one or more fruits, total number of grams of one or more supplements, probiotics or vitamins, etc. In another example, the element may include an amount of (AGEs) in a portioned meal or set of meals. The AGE levels may be measured in the ingredients, or in the finished product. In some cases, an Inflammation Index may be provided on the package of the portioned meal. In some cases, the desired AGEs level in a nutritional plan may be adjusted based on a measure of the (AGEs) in the animal’s body. For example, a medical professional may revise or modify the nutritional plan particularly about the AGEs level based on the biological samples from animal’s body (e.g., blood, tissue, microbiome, etc.). This may beneficially reduce the inflammation load further reduces systemic disease.

The nutritional plan may provide personalized meals on a per-meal basis or partial serving size basis. For example, the nutritional plan may breakdown the nutritional requirements for an animal on a weekly, daily, full serving size, additional serving size and/or partial serving size basis, or another cadence appropriate for a pet. For example, the nutritional plan may evenly divide the nutritional requirements up for a particular week by day and/or meal during that week, or the nutritional plan may vary the nutrition provided in each meal based on meal type. For example, the nutritional value provided during the morning meal, the midday meal, the evening meal, and snack(s) may vary. The types of meal may also include treats. For example, the treat may also be customized and include nutritionally complete and balanced ingredients. In some cases, the nutritional value for meals provided within a day may vary depending on an activity habit of the animal.

The nutritional value of each meal may be based on goals set by the veterinarian and the condition of the animal as provided in the animal characteristics. For example, if the animal characteristics indicate a skin condition, then the nutrition provided in the meals may include ingredients to address the skin condition or if the animal’s target weight or body composition is different than the animal’s current weight or body composition, then the total calories provided to the animal may be greater or less than the number of calories in a weight or composition maintaining nutritional plan. In some embodiments, the animal characteristics may include a target weight loss or gain rate or in some embodiments a target weight loss or gain rate may be determined at block 130. Based on target weight loss or gain rate, a total daily caloric input for the animal may be determined.

In some embodiments, the individual nutritional plan may also be based on medical outcomes, for example, diagnostic testing or other blood analysis may show that the animal’s taurine level is 100, whereas a more suitable blood taurine level is 200, accordingly, the nutritional plan may vary to increase the taurine level to 200, or DEXA (dual energy. x-ray. absorptiometry). Similarly, other treatments may be part of the nutritional plan, such as treating diseases or conditions with medication administered through food.

The back end nutritional system 220, depicted in FIG. 2, may carry out the actions of block 130. For example, in some embodiments after receiving the animal characteristics, a backend system compares the animal characteristics with known nutritional data for animals. Based on the characteristics and known nutritional data, the backend system may then determine the nutritional plan for the animal. The nutritional plan may include a target range of values or target value for each of the nutritional factors, such as calories, grams of fat, etc. After determining the nutritional plan for the animal, the method may proceed to block 140.

In some embodiments, the nutritional plan may be generated to facilitate a transition from a previous pet food to a newly recommended nutritional plan. For example, the nutritional plan may include a set of meals designed to be provided in combination with the previous food with a gradual reduction in the previous food and gradual increase in the new nutritional meals. The set of new meals may be portioned in accordance with a recommended portion for the previous pet food to facilitate a transition to the new nutritional plan.

In some embodiments, the individual nutritional plan may be generated using a model or algorithms. The model or algorithm may be pre-determined based on empirical data. In some cases, the model may be a trained model. For instance, the nutritional system may employ one or more machine learning trained classifiers or trained predictive models to take one or more animal’s characteristics and goals as input, and generate a predicted nutritional plan as output.

The predictive models may be trained and developed by the nutritional system and make inferences on the cloud. Alternatively, the predictive models may be trained, developed and built on the cloud and is downloaded to a third-party system (e.g., manufacturing system, veterinary pratice) or executed by the third-party system for inference. A predictive model may be a trained model or trained machine learning algorithm. The machine learning algorithm can be any type of machine learning network such as: a support vector machine (SVM), a naive Bayes classification, a linear regression model, a quantile regression model, a logistic regression model, a random forest, a neural network, convolutional neural network CNN, recurrent neural network RNN, a gradient-boosted classifier or repressor, or another supervised or unsupervised machine learning algorithm (e.g., generative adversarial network (GAN), Cycle-GAN, etc.

In some cases, the predictive models may be continually trained or improved after deployment. The predictive model provided by the system may be dynamically adjusted and tuned to adapt to different users, veterinary systems, and data over time. The predictive model provided by the platform may be improved continuously over time (e.g., during implementation, after deployment). Such continual training and improvement may be performed automatically with little user input or user intervention.

At block 140 individualized meals are determined based on the nutritional plan. Various available ingredients for each meal’s contents and their nutritional values may be stored in a database and retrieved therefrom. In some embodiments, the number of meals an animal is provided each day or week also may be individualized, such that a pet’s nutritional recommendation could change on a per meal basis, for example, from 3 meals of 500 calories each to 2 meals of 750 calories each.

In some cases, the individualized meals may be adjusted per meal-specific calories/content simultaneously, based on the feedback from the veterinary professional or nutritionist, or feedback from a pet owner. For example, a veterinary professional or nutritionist may provide feedback via a user interface to adjust one or more meals in a nutritional plan based on a biological sample analysis result, or diagnostic tests. In some cases, the feedback may also include information indicative of how well the animal digested the food or sample collection for digestion tests. For example, an analysis may be performed on a collected biological sample to examine a health and digestive indicator.

A pet owner may also be permitted to provide feedback after providing the food to the animal. For example, the pet owner may input information related to weight or the pet, change in activity level, or the pet reaction to a meal via the user interface which may be utilized to further adjust the meals. The feedback information may be provided at pre-determined check point. For instance, the user application may prompt the pet owner to follow up and provide feedback information daily, weekly or monthly. Alternatively, the pet owner may provide the feedback at any time. The feedback information may be provided in any suitable form. For example, the pet owner may provide the feedback via a graphical user interface (GUI) provided on a user device by uploading an image of the animal or a sample (e.g., waste) of the animal, a video of the animal, filling a form or survey and the like. In some cases, the user application may also collect motion data from the tracking device and analyze an activity level of the animal. The input information such as the image, video or motion data may be analyzed to extract information indicating enjoyment of the food, time spent at the bowl, speed of eating, position of the head, ease of chewing, and the like. In some cases, deep learning techniques may be employed to extract such insight information. For instance, the video or image may be processed using a trained model to predict a enjoyment level of the food.

At block 140, the backend system selects, based on user input and/or analytics, ingredients from the available ingredients for each meal. The system may select a combination of ingredients such that the resulting nutritional value provided by each meal is within the target range for each meal. Ingredients may include AAFCO or other defined ingredients, including supplements and additives and may also include non-nutritional elements, such as pharmaceutical or other prescribed drugs for treating diseases. Ingredients may also include air dried or low AGE (Advanced Glycation End Products) dry kibble or nutritional ingredients of the meals may also be fermentation based, high pressure process based, or humectant based preservation ingredients.

In some embodiments the target range may be set on a daily and serving basis. This may advantageously provide flexibility to determine ingredients regardless the form or serving size of the available ingredients. For example, the ingredients may be pelletized, and accordingly, the system may not be able to match the value of the meal with the target range or target value. In such embodiments, the backend system may provide less of a particular nutritional characteristic in one meal of the day and compensate with more of the nutritional characteristic in another, different, meal for that day. Similarly, the system may compensate on a weekly basis or some other cadence, as appropriate.

In some embodiments, the nutritional system may gather the available ingredients (e.g., type of ingredients, serving size/unit of ingredients, form of ingredients, etc.) from application programming interfaces (APIs) or third-party resources (e.g., manufacturer system, ingredients inventory). For example, the nutritional system may utilize API requests to interact with manufacturer systems to aggregate those ingredients information for determining individual meals or interact with a veterinary practice to receive updated animal characteristics or input from a medical professional.

In some cases, the ingredients for each meal may be generated using a trained model. For instance, the nutritional system may employ one or more machine learning trained classifiers or trained predictive models to take in information about the available ingredients and nutritional plan as input, and generate the ingredients for a given meal as output.

The ingredients for each meal are then sent to a manufacturing system, such as manufacturing system 230, 400 for production of the meals. In some cases, feedback may also be collected to adjust the ingredients. For example, information indicative of how well the animal digested the food or sample collection for digestion tests may be obtained to adjust the ingredients. For example, an analysis may be performed on a collected biological sample to examine a health and digestive indicator and the ingredients may be adjusted based on the digestive indicator.

At block 150 the individualized meals are fabricated. In some embodiments, the meals are fabricated using a manufacturing system, such as manufacturing systems 230, 400. At block 150, each of the ingredients are then dispensed into a package for each meal. The package is then sealed for transportation and provided to the veterinarian or animal owner. In some embodiments, the packaging may use compostable or consumable bags to avoid excess waste. Details about the manufacturing system are described later herein. In some cases, a package may include ingredients for multiple meals of the same meal type (e.g., morning meal, snack, etc.). In such cases, measuring cup or marked bowl may be utilized to portion the pre-determined amount of food.

FIG. 2 illustrates a system 200 for individually planning an animal’s meals and the various connections between each of the elements of the system 200. The system 200 includes one or more veterinary practice systems 210a, 210b, a backend system 220, and a manufacturing system 230.

The veterinary practice systems or applications 210a, 210b may include one or more systems for collecting, storing, and transmitting animal characteristics. The veterinary practice systems or applications may include one or more objects that are the same as those described in US 10013530 which is incorporated by reference herein in the entirety. For example, the respective veterinary practice system may comprise a respective data integration agent and browser, each of which can independently communicate data using the communication path. The communication path enables communication with a plurality of programmatic elements in the backend nutritional system 220. In some cases, the backend nutritional system may comprise a web server and online services. The web server may include a web user interface configured to exchange information between the computing devices of the respective veterinary practices and the backend nutritional system 220. Each of the computing devices can receive information from users in a practice information management system and communicate with the respective data integration agent of the computing device. The data integration agent thus provides a connection between the user information provided to the practice information management system and the backend nutritional system 220 via the network.

In some cases, the data integration agent may be provided by the nutrient system 220 and may be installed in the computing device of the veterinary practice. The data integration agent is a system which integrates with these varied systems to provide added value and operational simplicity for employees of the veterinary practice and pet owners. The data integration agent may be responsible for retrieving and mapping data from the PIMS (Practice Information Management System), sending communications to and receiving information from the veterinary system about animal medical records, diagnostic result and the like, and communication with the browsers to receive information such as a nutrient goal or feedback. The data integration agent may employ various technological mechanisms to limit traffic between the components of the network, creating efficient correspondence between all systems.

In some cases, the data integration agent may include an abstraction engine that allows communication with various PIMS systems, as well as the ability to integrate with additional in the future in an ad-hoc fashion. For example, the data abstraction engine may provide a data abstraction layer over any databases, storage systems, and/or the stored data that has been stored or persisted by the systems. The data abstraction layer can include various components, subsystems and logic for translation standards and mappings to translate the various incoming database access requests into the appropriate queries of the underlying databases. For instance, the data abstraction layer is located between an application (e.g., nutritional application, PIMS application or other cloud applications) and the underlying physical data. The data abstraction layer may define a collection of logical fields that are loosely coupled to the underlying physical mechanisms (e.g., database) storing the data. The logical fields are available to compose queries to search, retrieve, add, and modify data stored in the underlying database. This beneficially allows the nutritional system to communicate with varieties of databases or storage systems via a unified interface.

In some cases, the veterinary practice system 210a, 210b may include one or more computing devices. Each computing device may be used by (or integrated into) a veterinary practice and allow the veterinary practice to interact, collect, store, and transmit animal characteristics. Each computing device may include a processor based device with storage, memory, a display and wireless or wired connectivity circuits that allow the computing device to interact with the veterinarian and the backend nutritional system. For example, each computing device may be a smartphone device, such as a device operating using the iOS, Android or Symbian operating systems, a personal computer, a client/server system, a terminal, a tablet computer, a cellular phone and any other device that would be capable of interacting with the backend nutritional system. In one implementation, one or more of the computing devices may include a practice information management system that interacts with the backend nutritional system.

In one implementation, the practice information management system may be a plurality of lines of code executed by the processor of the computing device. In some implementations, each of the computing devices may have a browser that interacts with the practice information management system, displays web pages and allows the user (e.g., medical professional) to enter information into forms or via a user interface for transmission to the practice information management system. In one implementation, the browser may be a plurality of lines of computer code executed by the processor of the computing device.

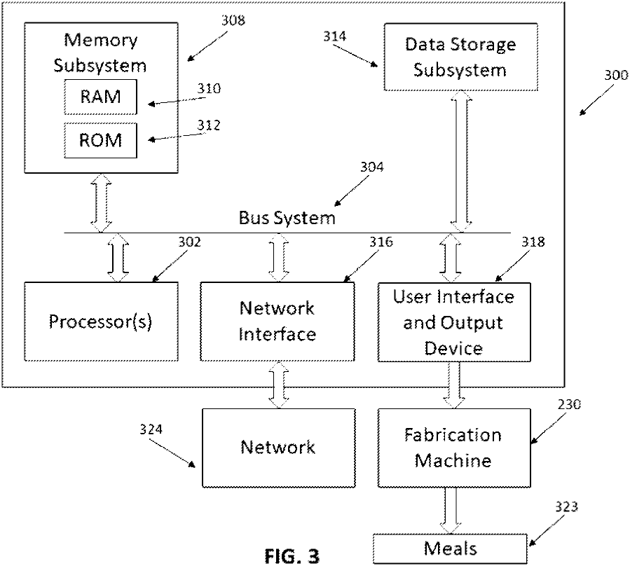

Backend System 220 is shown and described in more detail with respect to FIG. 3. FIG. 3 is a simplified block diagram of a computer system implementing the backend system, which may be the same as manufacture system 230 and may be used in executing methods and processes described herein. The data processing system 300 typically includes at least one processor 302 that communicates with one or more peripheral devices via bus subsystem 304. These peripheral devices typically include a memory subsystem 308 and data storage subsystem 314, a set of user interface input and output devices 318, and an interface to outside networks 316. This interface is shown schematically as “Network Interface” block 316, and is coupled to corresponding interface devices in other data processing systems via communication network 324. The backend system 220 communicates with the veterinary systems via then network 324. Data processing system 300 can include, for example, one or more computers, such as a personal computer, workstation, mainframe, laptop, and the like.

The user interface input devices 318 are not limited to any particular device, and can typically include, for example, a keyboard, pointing device, mouse, scanner, interactive displays, touchpad, joysticks, etc. Similarly, various user interface output devices can be employed in a system of the invention, and can include, for example, one or more of a printer, display (e.g., visual, non-visual) system/subsystem, controller, projection device, audio output, and the like.

Data storage subsystem 314 maintains the basic required programming, including computer readable media having instructions (e.g., operating instructions, etc.), and data constructs. The program modules discussed herein are typically stored in the data storage subsystem 314. Data storage subsystem 314 provides persistent (non-volatile) storage for program and data files, and can include one or more removable or fixed drives or media, hard disk, floppy disk, CD-ROM, DVD, optical drives, flash or USB drives, cloud-based storage, and the like. One or more of the storage systems, drives, etc., may be located at a remote location, such coupled via a server on a network or via the internet/World Wide Web. In this context, the term “bus subsystem” is used generically so as to include any mechanism for letting the various components and subsystems communicate with each other as intended and can include a variety of suitable components/systems that may be known or recognized as suitable for use therein. It will be recognized that various components of the system can be, but need not necessarily be at the same physical location, but may be connected via various local-area or wide-area network media, transmission systems, etc.

Memory subsystem 608 typically includes a number of memories (e.g., RAM 310, ROM 312, etc.) including computer readable memory for storage of fixed instructions, instructions and data during program execution, basic input/output system, etc.

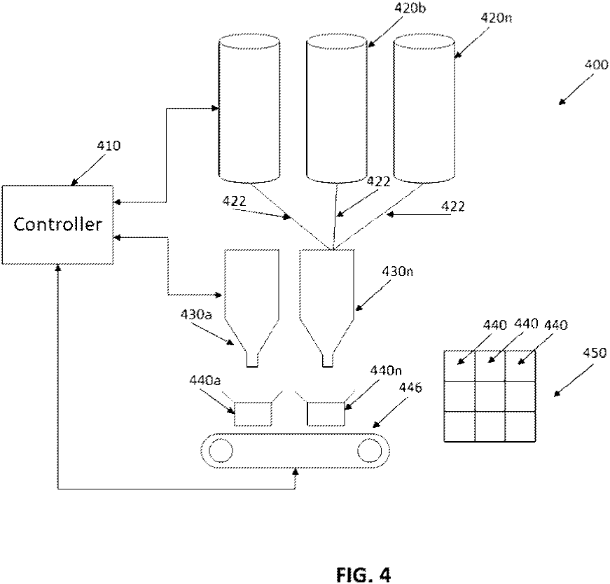

FIG. 4 schematically illustrates a fabrication/manufacturing system 400. The fabrication/manufacturing system 400 can be the same as the manufacture system 230 in FIG. 2 and FIG. 3. For example, the manufacture system fabricates the meals 323, 440 based on the individualized meal plans determined at block 140 of method 100 and sent by the backend nutritional system 220 to the fabrication machine 323, 400. The fabrication machine 400 can, for example, be located at a remote location and receive data from backend nutritional system 300 via network 324.

In some embodiments, the fabrication system 400 may comprise a controller 410, ingredient storage 420a, ingredient metering and dispensing units 430a, ingredient transport 422, and a meal transport 446. During operation, the controller 410 receives the individualized meal information from the backend nutritional system 220 and then causes the meals to be produced and packaged. The controller communicates with or otherwise controls the various components of the system 400.

The ingredient storage containers 420 include segregated storage for each of the different ingredients for use in the individualized meals 440a. For example, ingredient storage container 420a may include the bulk nutritional product that forms the basis for the individualized meal. This may include the basic ingredients that make up most or all of the meals made by the manufacturing system 400. Ingredient storage container 420a may include supplements, for example, protein supplements for individualized meals that call for more protein. Other ingredient storage containers may include supplements to treat animal condition or diseases, such as skin conditions. In some embodiments, ingredient storage containers may be made with compostable or consumable materials to avoid excess waste.

When manufacturing an individualized meal, the ingredients from the various ingredient storage containers 420 may be transported to the ingredient metering and dispensing units 430a, 430n by one or more ingredient transports 422. The ingredient transports may be augers, conveyers, pneumatic, belts, buckets, extruders or other transport systems that move the ingredients to the one or more ingredient metering and dispensing units 430a, 430n.

The ingredient metering and dispensing units 430a, 430n may automatically meter and dispense the appropriate amount of each ingredient into the individualized meal package 440a. The metering and dispensing unit may include an auger, conveyor or extruder dispensing system that accurately dispenses the ingredients into the individualized meal packages 440a, 440n. The units 430a may include one or more measuring devices to measure the amount of each ingredient dispensed into the package 440a. For example, the units 430a may include a volumetric measuring device to accurately measure the volume of ingredients dispensed. In some embodiments, the units 430a may include a mass measuring device that measures the mass of the ingredients dispensed into the package 440a. The measuring device may be connected to the controller 410, such that when the appropriate amounts of ingredients have been dispensed, the controller turns the unit 430a off.

In some embodiments, the ingredient metering and dispensing units 430a may also act as a storage unit (intermediate storage for blending or coating and finished product). For example, ingredient metering and dispensing unit 430a is not supplied by a separate storage unit 420b, 420n. Instead, ingredient metering and dispensing unit 430a may be batch filled or may include a single use container of an ingredient that is then directly dispensed into the individualized meal packages 440a, 440n. Although ingredient metering and dispensing unit 430a, 430n is depicted as receiving ingredients from several storage containers 420b, 420n, in some embodiments, each ingredient metering and dispensing unit 430a, 430n may be connected to a single storage container 420b, 420n.

The meal transport 446 may be a conveyer that moved the individualized meal packages 440a, 440n to each ingredient metering and dispensing unit 430a, 430n to receive the ingredients. The meal transport 446 may transport the individualized meal packages to a sealing machine and then on to final packaging. At final packaging each individualized meal is packaged into a meal set 450. A meal set 450 may be a set of meals 440 for a particular period of time, such as a week or month of meals for an animal. The set of meals 440 may be different according to the nutritional plan. A meal set 450 may include, for example, individually packaged meals for a day. An individually packaged meal in the meal set 450 may include the portioned combination of ingredients determined for the meal.

After packaging into a meal set 450, the meal set is sent to the veterinarian’s office or to a pet owner for provision to the animal by the animal’s care taker. In some cases, once a meal set if shipped, a notification may be delivered to the pet owner.

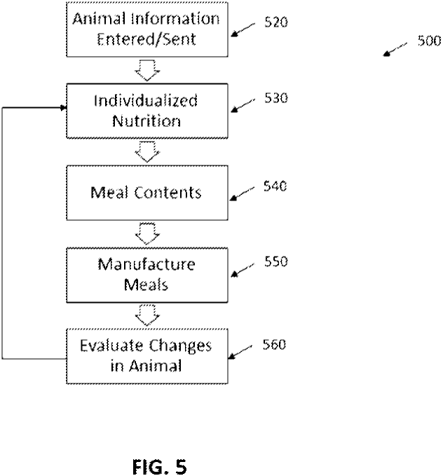

In some embodiments, after receiving and consuming one or more meal sets, the pet owner, veterinarian or veterinarian staff member updates the animal characteristics, at least in part based on the changes to the animal. These updates characteristics are sent to the backend system and new individualized meals are prepared based on the method 100. In addition, the plan may change based on changes to the animal’s characteristics, such as adjusting the nutrition based on age, as the animal ages or lifestyle changes, activity level, or based on health changes, such as developing a diseases or allergy. FIG. 5 shows an example of such a method.

The method 500 may begin at block 520, wherein animal characteristics for an animal are received, similar to block 120 of FIG. 1. At block 520 an animal’s characteristics may be received from a veterinary office. These animal characteristics may include the animal’s age, sex, breed, gender, species, weight, height, mature body weight, body condition score, muscle condition score, diseases, conditions, blood analysis results, medical history, current treatments, activity level, supplements and/or medications the animal is taking or prescribed, ideal weight, activity level and etc. The animal’s characteristics may be received from a veterinarian or veterinarian system, such as veterinarian system 210a, depicted in FIG. 2. In some embodiments, mobile or other telemedicine applications that use audio, stored video or live feeds maybe used to gather information about the pet’s characteristics such as the animal’s weight/condition and other characteristics described herein.

At block 530, an individualized nutritional plan for each animal is determined, similar to block 130 of FIG. 1. The individualized nutritional plan is determined based on the characteristics of the animal, which may include historic, current and anticipated future characteristics. The individualized nutritional plan may also be determined based on known nutritional guidelines for particular species and breeds of animals. The individualized nutritional plan may include one or more elements. For example, the individualized nutritional plan may include total caloric intake, total number of calories from one or more carbohydrates, total number of calories from one or more fats, total number of calories from one or more proteins, total number of calories from one or more vegetables, total number of calories from one or more fruits, total number of grams of one or more fats, total number grams of one or more proteins, total number of grams of one or more vegetables, total number of grams of one or more fruits, total number of grams of one or more supplements, probiotics or vitamins, etc. The nutritional plan may breakdown the nutritional requirements for an animal on a weekly, daily, full serving size, additional serving size and/or partial serving size basis. For example, the nutritional plan may evenly divide the nutritional requirements up for a particular week by day and/or meal during that week, or the nutritional plan may vary the nutrition provided in each meal based on meal type (e.g., morning meal, the midday meal, evening meal, snack, etc.). For example, the nutritional value provided during the morning meal, the midday meal, the evening meal, and a snack may vary.

The back end nutritional system 220, depicted in FIG. 2, may carry out the actions of block 530. For example, in some embodiments after receiving the animal characteristics, a backend system compares the animal characteristics with known nutritional data for similar individual animals. Based on the characteristics and known nutritional data, the backend system may then determine the nutritional plan for the animal. The nutritional plan may include a target range of values or target values for each of the nutritional factors, such as calories, grams of fat, etc. After determining the nutritional plan for the animal, the method may proceed to block 140.

At block 540 individualized meals are determined based on the nutritional plan, similar to block 140 of FIG. 1. Various available ingredients for each meal’s contents and their nutritional values may be stored in a database and retrieved therefrom. At block 140, the backend system selects ingredients from the available ingredients for each meal. The system may select a combination of ingredients such that the resulting nutritional value provided by each meal is within the target range for each meal. Ingredients may include AAFCO defined ingredients, including supplements and additives and may also include non-nutritional elements, such as pharmaceutical or other prescribed drugs for treating diseases. Ingredients may also include air dried or low AGE dry kibble or nutritional ingredients of the meals may also be fermentation based, high pressure processed based, or humectant based preservation ingredients.

At block 550 the individualized meals a fabricated, similar to block 150 of FIG. 1. In some embodiments, the meals are fabricated using a manufacturing system, such as manufacturing systems 230, 400. At block 150, each of the ingredients are then dispensed into a package for each meal. The package is then sealed for transportation and provided to the veterinarian or animal owner.

In some embodiments the target range may be set on a daily and meal-by-meal basis. In some embodiments, the ingredients are pelletized, and accordingly, the system may not be able to match the value of the meal with the target range or target value. Some owners may choose for one meal of the day to be larger or smaller than another meal for other considerations unrelated to nutrition (e.g., a pet owner may not be able to walk the pet during the day or at night, or a pet owner may have a varied exercise program for a pet such as in cases where the pet is providing a service, etc.). In such embodiments, the backend system may provide less of a particular nutritional characteristic in one meal of the day, week or month, or other cadence, and compensate with more of the nutritional characteristic in another, different, mean for that day, week or month or other. The ingredients for each meal are then sent to a manufacturing system, such as manufacturing system 230, 400 for production of the meals.

At block 560, after the animal has been consuming the meals according to the nutritional plan for a period of time, such as one week, one month, three months, six months, a year, etc., the animal is evaluated by the veterinarian or veterinarian professional, such as a veterinarian technician or other professional involved in the care and treatment of an animal, either during a special visit scheduled to evaluate nutrition or during a regular checkup. Such checkup may focus on assessing the wellness of the animal. Here, the veterinary care provider updates the animal characteristics for the animal. The veterinary care provider may update the animal’s age, weight, height, lifestyle, diseases, conditions, microbiome, blood analysis results, medical history, current treatments, supplements and/or medications the animal is taking or prescribed, ideal weight, and etc.

In some cases, once the animal has begun consuming the meals according to the nutritional plan, the system may automatically track the status of the animal and generate an alert or notification upon detection of non-compliance event or other events. A warning or notification may be triggered and sent to the medical professional or the pet owner for example upon detection of non-compliance to the nutritional plan (e.g., incorrect feeding time, incorrect types of meals being served), or a tracked health status of the animal (e.g., an abnormal activity level, digestive problem, etc.). This may beneficially allow for the medical professional to intervene to adjust the nutritional plan in a timely fashion and/or for the pet owner to correct the feeding to be compliant with the nutritional plan. In some embodiments, the detection of a non-compliance event and/or abnormal status may be enabled by deep learning techniques as described elsewhere herein. For example, sensor data (e.g., motion sensor) or images captured by the tracking device or user device may be processed by a trained model to detect the event.

The animal’s characteristics may be received from a veterinary care provider or veterinarian system, such as veterinarian system 210a, depicted in FIG. 2. The veterinarian system 210a may include a practice information management system, or PIMS, that includes the animal characteristics. The animal’s characteristics may be received by a nutritional system, such as the backend nutrition system 220, depicted in FIG. 2.

In some embodiments, a veterinarian or veterinarian professional, such as a veterinarian technician or other professional involved in the care and treatment of an animal enters the information into their veterinary practice information management system, which may be part of the veterinary system 210. In some embodiments, a pet owner or care taker or service provider can adjust the data using an application on their own device, for example, between veterinary visits. In some embodiments, the changes that may be made by an animal owner or care taker may be limited by a veterinarian or veterinarian professional, or the data may be collected but used differently than data stored within the PIMS. The veterinary practice information management system may provide the animal characteristics to the backend and nutritional system 220, depicted in FIG. 2.

After updating the animal’s characteristics, the process may proceed to block 530 again for development of an individualized nutrition plan and the process continues.

The data processing aspects of the methods described herein can be implemented in digital electronic circuitry, or in computer hardware, firmware, software, or suitable combinations thereof. Data processing apparatus can be implemented in a computer program product tangibly embodied in a machine-readable storage device for execution by a programmable processor. Data processing blocks can be performed by a programmable processor executing program instructions to perform functions by operating on input data and generating output. The data processing aspects can be implemented in one or more computer programs that are executable on a programmable system, the system including one or more programmable processors operably coupled to a data storage system. Generally, a processor will receive instructions and data from a read-only memory and/or a random access memory. Storage devices suitable for tangibly embodying computer program instructions and data include all forms of nonvolatile memory, such as: semiconductor memory devices, such as EPROM, EEPROM, and flash memory devices; magnetic disks such as internal hard disks and removable disks; magneto-optical disks; and CD-ROM disks. Data streams are then integrated for better decision making by the veterinarian, staff member or other advisor, in consultation with the pet owner.

The system and method disclosed herein may be implemented via one or more components, systems, servers, appliances, other subcomponents, or distributed between such elements. When implemented as a system, such systems may include and/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection; however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, and can include one or more removable or fixed drives or media, floppy disk, CD-ROM, DVD, optical drives, flash or usb drives, cloud-based storage, and the like, etc.) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media.

FIG. 6 shows a network environment 600 in which a nutritional information exchange system is implemented. A network environment 600 may include one or more user devices 601, a tracking device 605, a nutritional information exchange system 610, one or more manufacture systems 630, veterinary systems 620, and a database 615, 621, 641. Each of the components may be operatively connected to one another via a network 650 or any type of communication link that allows transmission of data from one component to another.

In some embodiments, the nutritional information exchange system 610 may include one or more components such as a nutrition planning module 611, user interface (UI) module 613, data storage and processing module or other cloud applications. The nutritional information exchange system 610 can be the same as the nutritional system as described above. For example, the nutritional information exchange system 610 may be implemented as one or more computing resources or hardware devices. The nutritional information exchange system 610 may be implemented on one or more server computers, one or more cloud computing resources and the like and each resource has one or more processors, memory, persistent storage and the like. For instance, the nutritional information exchange system 610 may comprise a web server, online services, nutrition planning module, UI module and the like for providing nutrition applications to pet owners 603 and/or veterinary practices 623. For instance, a web server may be implemented as a hardware web server or a software implemented web server, may generate and exchange web pages with each computing device 601, 620 that is using a browser.

In some cases, the nutrition planning module 611 may be configured to generate nutritional plans and individualized meals as described elsewhere herein. For example, the nutrition planning module 611 may employ the machine learning techniques to generate and/or adjust a nutritional plan and/or individualized meals based on animal characteristics and/or feedback.

The individual meals and ingredients for each meal generated by the nutrition planning module 611 may be transmitted to the manufacture system 630. The manufacture system can be the same as the fabrication/manufacturing system as described in FIG. 4. For example, the controller 630 may receive the nutrient ingredients for each meal and control the fabrication machine 631 to produce and package a meal set as described elsewhere herein.

The nutrition planning module 611 may employ any suitable technologies such as container and/or micro-service. For example, the nutrition planning module 611 may be implemented as cloud applications that can be a containerized application. The nutrition planning module may deploy a micro-service based architecture in the software infrastructure such as implementing a nutrition planning application or service in a container.

In some cases, the UI module 613 and the nutrition planning module 611 may include software applications (i.e., client software) for veterinary practices 621 and pet owner 603 allowing for exchanging information between the hospital, pet owner and the nutritional information exchange system 610. For example, applications running on the hospital/veterinary practice device (e.g., client/browser) may allow inputting nutrition goals, modifying a nutrition plan, reviewing nutrition plans, searching PIMS data for clients and the like. In some cases, the nutrition interfaces or APIs may be integrated to a current mobile application running on the pet owner device 601 and/or integrated into a current front-end user interface (e.g., within the GUI) running on the veterinary practice device 620. The current user interfaces may be hosted by a separate server.

The applications provided by the nutritional information exchange system may be cloud-powered applications or local applications. The nutrition planning module and UI module may also provide software applications (i.e., client software) for pet owners 603. The client applications may allow pet owners to enroll in a nutrition plan service, track the status of meals, record or input health status of pets, and the like.

In some embodiments, the UI module may generate one or more graphical user interfaces (GUIs) for the pet owner interface running on the pet owner device 601 and for the medical professional running on the veterinary system 620. The GUIs may be rendered on a display screen on a user device (e.g., a participant device) 601, 620. A GUI is a type of interface that allows users to interact with electronic devices through graphical icons and visual indicators such as secondary notation, as opposed to text-based interfaces, typed command labels or text navigation. The actions in a GUI are usually performed through direct manipulation of the graphical elements. In addition to computers, GUIs can be found in hand-held devices such as MP3 players, portable media players, gaming devices and smaller household, office and industry equipment. The GUIs may be provided in software, a software application, a mobile application, a web browser, or the like. The GUIs may be displayed on a user device (e.g., desktop computers, laptops or notebook computers, mobile devices (e.g., smart phones, cell phones, personal digital assistants (PDAs), and tablets), and wearable devices (e.g., smartwatches, etc.).

The tracking device 605 may be in communication with the user device via a local communication channel, or with the backend system 610 via the network 650. The tracking device 650 may be attached to the animal 607, and may wirelessly communicate with the backend system 610 or the user device 601. In some embodiments, the tracking device may include an animal tracking mechanism that tracks motion or a current location of the animal, a kinetic motion energy generator electrically connected to the animal tracking mechanism, the kinetic motion energy generator capable of generating electrical energy in response to a normal movement of the animal and the animal tracking mechanism having an energy store that powers the animal tracking mechanism. In some cases, the energy store is powered by the electrical energy generated by the kinetic motion energy generator.

The tracking device may include one or more sensors to collect data about a motion of the animal to determine an activity level. The tracking device may include various types of sensors such as physiologic sensors, kinematic sensors, audio sensors and the like to track an activity level, or health condition of the animal. Examples of types of sensors may include inertial sensors (e.g., accelerometers, gyroscopes, and/or gravity detection sensors, which may form inertial measurement units (IMUs)), location sensors (e.g., global positioning system (GPS) sensors, mobile device transmitters enabling location triangulation), heart rate monitors, external temperature sensors, skin temperature sensors, skin conductance, neural signals (e.g. EEG), muscle signals (e.g. EMG), sensors configured to detect a galvanic skin response (GSR), proximity or range sensors (e.g., ultrasonic sensors, lidar, time-of-flight or depth cameras), altitude sensors, attitude sensors (e.g., compasses), pressure sensors (e.g., barometers), humidity sensors, vibration sensors, audio sensors (e.g., microphones), and/or field sensors (e.g., magnetometers, electromagnetic sensors, radio sensors).

The data storage and processing module 615 may comprise at least data input module and a data integration agent as described above for enabling data transmission between the nutritional information exchange system 610 and other components of the network 600. The data integration agent may be deployed to the veterinary system 620 as a lightweight and native application to facilitate access to the database 621 and other data storage systems. The data input module may be configured to receive and pre-process input data.

The tracking device may comprise a controller powered by the energy from the battery and the electronic circuit and/or is programmed to manage the energy and functions of the device and the component and modules of the electrical component assembly. For example, the controller may be configured to switch between a high energy usage mode and a low energy usage mode. In the low energy usage mode a frequency and/or duration of the communication module of the device and/or the motion tracking mechanism may be further configured to provide that the net energy usage of the tracking device may be lower than or equal to the energy generated by the kinetic motion generator over a period of time. In the high energy usage mode a frequency and/or a duration of the communication module and/or the motion tracking mechanism may be further configured to provide a more frequent and/or longer duration of communication and/or location determination than in the lower energy usage mode.

In some cases, the input data received by the data input module may include data obtained from the databases 621, 641, 615, manufacture system 630, pet owners 603, motion tracking device 605, and/or a wide variety of sources. The input data may be related to one or more animal characteristics including structured data such as JavaScript object notation (JSON) data. In some cases, the input data may include unstructured data related to the animal characteristics, such as motion tracking data, image of pet, image of a sample, medical reports, emails, or web-based content. The unstructured input data such as motion data, email, or an image of a pet may be processed by the data input module to extract the animal characteristics prior to being processing by the nutrition planning module 611.

In some cases, the data input module may be in communication with one or more databases 621, 641 to retrieve relevant data. For instance, the data input module may retrieve the historical data (e.g., medical records, treatment history of the pet from any veterinary practice, data from other insurance providers, etc.) from a historical database based on the pet name, or pet identifier.

In some cases, the data input module may pre-process the input data to extract and/or generate the animal characteristic data to be processed by the nutrition planning module. In some cases, the data input module may employ a predictive model for extracting data features, natural language processing techniques or image recognition to extract health status data. For instance, a pet owner may take a picture of the pet or biological sample of the pet, the image may then be processed to assess a health status (e.g., digestive issues) or condition (e.g., weight, size, skin, etc.) of the pet. In some cases, the data input module may assemble the data received or retrieved from the varieties of data sources and convert the assembled dataset into a feature set to be processed by the nutrition planning module.

In some embodiments, the nutritional information exchange system may train, develop or test a predictive model using data from a cloud data lake (e.g., database 615). In some cases, the nutritional information exchange system may perform model deployment, maintenance, monitoring, model update, model versioning, model sharing, and various others. The nutritional information exchange system may also support ingesting data transmitted from the veterinary system, manufacture system and user device into one or more databases or cloud storages 615. In some cases, the received data may be used to generate training datasets (e.g., labeled data).

User device 601 associated with a pet owner and the user device 620 associated with a veterinary practice may be a computing device configured to perform one or more operations (e.g., rendering a user interface for inputting nutritional goals, modifications, information related to animal characteristics or medical information, reviewing nutritional plan or meal ingredients, etc.). Examples of user devices may include, but are not limited to, mobile devices, smartphones/cellphones, wearable device (e.g., smartwatches), tablets, personal digital assistants (PDAs), laptop or notebook computers, desktop computers, media content players, television sets, video gaming station/system, virtual reality systems, augmented reality systems, microphones, or any electronic device capable of analyzing, receiving (e.g., receiving image of animal, medical form, modification of fields of nutritional plan, etc.), providing or displaying certain types of data (e.g., nutritional plan, plot of nutrition, etc.) to a user. The user device may be a handheld object. The user device may be portable. The user device may be carried by a human user. In some cases, the user device may be located remotely from a human user, and the user can control the user device using wireless and/or wired communications. The user device can be any electronic device with a display.

User device may include a display. The display may be a screen. The display may or may not be a touchscreen. The display may be a light-emitting diode (LED) screen, OLED screen, liquid crystal display (LCD) screen, plasma screen, or any other type of screen. The display may be configured to show a user interface (UI) or a graphical user interface (GUI) rendered through an application (e.g., via an application programming interface (API) executed on the user device). The GUI may show current and historic nutritional plan, ingredients, ideal animal characteristics, nutrition goal, health status of an animal, interactive elements relating to a nutritional plan (e.g., editable fields, ingredients field, etc.). The user device may also be configured to display webpages and/or websites on the Internet. One or more of the webpages/websites may be hosted by server and/or rendered by the nutritional information exchange system 610 as described above.

User devices 601 may be associated with one or more users (e.g., pet owners). In some embodiments, a user may be associated with a unique user device. Alternatively, a user may be associated with a plurality of user devices. A user (e.g., pet owner) may be registered with the nutritional information exchange system. In some cases, for a registered user, user profile data may be stored in a database (e.g., database 615, 641) along with a user ID uniquely associated with the user. The user profile data may include, for example, pet name, pet owner name, geolocation, contact information, historical data, and various others as described elsewhere herein. In some cases, a registered user may be requested to log into the nutritional planning account with a credential. For instance, in order to perform activities such as requesting a personalized meal plan or submitting feedback to adjust a nutritional plan, a user may be required to log into the application by performing identity verification such as providing a passcode, scanning a QR code, biometrics verification (e.g., fingerprint, facial scan, retinal scan, voice recognition, etc.) or various other verification methods via the user device 601.

Network 650 may be a network that is configured to provide communication between the various components illustrated in FIG. 6. The network may be implemented, in some embodiments, as one or more networks that connect devices and/or components in the network layout for allowing communication between them. Direct communications may be provided between two or more of the above components. The direct communications may occur without requiring any intermediary device or network. Indirect communications may be provided between two or more of the above components. The indirect communications may occur with aid of one or more intermediary device or network. For instance, indirect communications may utilize a telecommunications network. Indirect communications may be performed with aid of one or more router, communication tower, satellite, or any other intermediary device or network. Examples of types of communications may include, but are not limited to: communications via the Internet, Local Area Networks (LANs), Wide Area Networks (WANs), Bluetooth, Near Field Communication (NFC) technologies, networks based on mobile data protocols such as General Packet Radio Services (GPRS), GSM, Enhanced Data GSM Environment (EDGE), 3G, 4G, 5G or Long Term Evolution (LTE) protocols, Infra-Red (IR) communication technologies, and/or Wi-Fi, and may be wireless, wired, or a combination thereof. In some embodiments, the network may be implemented using cell and/or pager networks, satellite, licensed radio, or a combination of licensed and unlicensed radio. The network may be wireless, wired, or a combination thereof.

User devices 601, veterinary practice computer system 620, nutritional information exchange system 610 and manufacture system 630, may be connected or interconnected to one or more database 621, 641, 615. The databases may be one or more memory devices configured to store data. Additionally, the databases may also, in some embodiments, be implemented as a computer system with a storage device. In one aspect, the databases may be used by components of the network layout to perform one or more operations consistent with the disclosed embodiments. One or more local databases, and cloud databases of the platform may utilize any suitable database techniques. For instance, structured query language (SQL) or “NoSQL” database may be utilized for storing the nutrition data, pet/user profile data, historical data, predictive model, training datasets, or algorithms. Some of the databases may be implemented using various standard data-structures, such as an array, hash, (linked) list, struct, structured text file (e.g., XML), table, JavaScript Object Notation (JSON), NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. In some embodiments, the database may include a graph database that uses graph structures for semantic queries with nodes, edges and properties to represent and store data. If the database of the present invention is implemented as a data-structure, the use of the database of the present invention may be integrated into another component such as the component of the present invention. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A system for nutritional information exchange comprising:
a backend component implemented on a computer, wherein the backend component is configured to:
(a) receive data from a system in a veterinary practice via a data integration agent, wherein the data comprises information about one or more characteristics of an animal and a nutrition goal;
(b) create a nutritional plan based on the data, wherein the nutritional plan includes a set of meals each comprising one or more elements varied based at least in part on a type of meal;
(c) select a portioned combination of ingredients for each of the set of meals based on (i) available ingredients received from a manufacture system and the (ii) one or more elements for each of the set of meals; and
(d) transmit the portioned combination of ingredients for each of the set of meals to the manufacture system for fabrication and packaging the portioned combination of ingredients for each meal individually.

2. The system of claim 1, wherein the system in the veterinary practice comprises a graphical user interface for receiving the data.

3. The system of claim 1, wherein the data integration agent is connected to the system in the veterinary practice, and wherein the data integration agent provides a data abstraction layer for enabling the backend component to access or retrieve the data from the veterinary practice.

4. The system of claim 1, wherein the one or more characteristics are extracted from the data using machine learning techniques.

5. The system of claim 1, wherein the backend component is configured to further receive activity data from a motion tracking device to obtain a characteristic about an activity level of the animal.

6. The system of claim 1, wherein the one or more characteristics of the animal comprise one or more of mature body weight, body condition score, muscle condition score, and ideal weight.

7. The system of claim 1, wherein the one or more elements comprise a nutritional value for a meal or the set of meals.

8. The system of claim 1, wherein the one or more elements comprise a nutritional value for a partial meal.

9. The system of claim 1, wherein the one or more elements comprise a target nutritional value range for a period of time.

10. The system of claim 1, wherein the type of meal includes a morning meal, a midday meal, an evening meal, a snack, or a treat provided within a day, or a subset thereof.

11. The system of claim 10, wherein a set of packaged meals include meals of different types to be provided within a day.

12. The system of claim 1, wherein the backend component is configured to further modify the nutritional plan based on a user input received from the system in the veterinary practice.

13. The system of claim 1, wherein the backend component is configured to further modify the nutritional plan based on data received from a user device associated with an owner of the animal.

14. The system of claim 13, wherein the data comprises an image of the animal.

15. The system of claim 1, wherein the nutritional plan is created, modified or supplemented using a machine learning algorithm trained model.

16. The system of claim 1, wherein the combination of ingredients is selected using a machine learning algorithm trained model.

17. Method for nutritional information exchange comprising:
(a) receiving data from a system in a veterinary practice via a data integration agent, wherein the data comprises information about one or more characteristics of an animal and a nutrition goal;
(b) creating a nutritional plan based on the data, wherein the nutritional plan includes a set of meals each comprising one or more elements varied based at least in part on a type of meal;
(c) selecting a portioned combination of ingredients for each of the set of meals based on (i) available ingredients received from a manufacture system and (ii) the one or more elements for each of the set of meals; and
(d) transmitting the portioned combination of ingredients for each of the set of meals to the manufacture system for fabrication and packaging the portioned combination of ingredients for each meal individually.

18. (canceled)

19. (canceled)

20. (canceled)

21. (canceled)

22. The method of claim 17, wherein the one or more characteristics of the animal comprise one or more of mature body weight, body condition score, muscle condition score, and ideal weight.

23. (canceled)

24. The method of claim 17, wherein the one or more elements comprise a nutritional value for a partial meal.

25. (canceled)

26. The method of claim 17, wherein the type of meal includes a morning meal, a midday meal, an evening meal, and a snack provided within a day, or a subset thereof.

27. (canceled)

28. (canceled)

29. (canceled)

30. (canceled)

31. (canceled)

32. The method of claim 17, wherein the combination of ingredients is selected using a machine learning algorithm trained model.

33. A system for nutritional information exchange comprising:
a nutritional management server in communication with one or more veterinary management systems and one or more manufacturing servers, wherein the nutritional management server comprises (i) a memory for storing a set of software instructions, and (ii) one or more processors configured to execute the set of software instructions to:
(a) receive and exchange, via a data integration agent, nutritional data from the one or more veterinary management systems, wherein the nutritional data comprises information about one or more characteristics of a selected animal and a nutrition goal and at least a portion of the nutritional data is retrieved from a database operably coupled to the one or more veterinary management systems;
(b) create a nutritional plan based at least in part on the nutritional data, wherein the nutritional plan includes a set of meals each comprising one or more elements varied based at least in part on a type of meal;
(c) select a portioned combination of ingredients for each of the set of meals based on (i) available ingredients received from the one or more manufacturing servers and the (ii) one or more elements for each of the set of meals; and
(d) transmit the portioned combination of ingredients for each of the set of meals to the one or more manufacturing servers for production and packaging of the portioned combination of ingredients for each meal individually for the selected animal.

34. The system of claim 33, wherein the nutritional management server further modifies the nutritional plan based on data received from at least one of a plurality of end node devices associated with the selected animal and an owner of the selected animal.
